(19) United States
(12) Patent Application Publication
(10) Pub. No.: US 2023/0257807 A1
(43) Pub. Date: Aug. 17, 2023

(54) EFFICIENT NUCLEIC ACID TESTING AND GENE SEQUENCING METHOD

(71) Applicant: SHANGHAI ADVANCED RESEARCH INSTITUTE, CHINESE ACADEMY OF SCIENCES, Shanghai (CN)

(72) Inventors: Zhongyang WANG, Shanghai (CN); Wenwen LI, Shanghai (CN); Kang XIAO, Shanghai (CN); Jing SUN, Shanghai (CN)

(21) Appl. No.: 18/026,950

(22) PCT Filed: Aug. 26, 2021
(86) PCT No.: PCT/CN2021/114636

§ 371 Date: Mar. 17, 2023

(30) Foreign Application Priority Data
Sep. 18, 2020 (CN) 202010982860.5

(51) Int. Cl. C12Q 1/6869; G01N 21/64; C12Q 1/6837; G06F 17/16

(52) U.S. Cl. CPC C12Q 1/6869; C12Q 1/6837; G01N 21/64; G06F 17/16; G01N 2021/6417; G01N 2021/6439

(57) ABSTRACT

The present invention provides an efficient nucleic acid testing and gene sequencing method. The method includes the steps of: S1: constructing a space and spectral calibration matrix A to serve as a prior information; S2: labeling a target nucleic acid sequence with fluorescent probes to prepare a nucleic acid chip having a spatial distribution, and exciting the nucleic acid chip with a light source to emit multicolor fluorescent signals, and sequentially modulating, encoding and collecting the multicolor fluorescent signals, and thus obtaining a fluorescence two-dimensional intensity measurement matrix Y; and S3: performing correlation calculation between the calibration matrix A and the measurement matrix Y through a correlation imaging algorithm, solving Y=AX, and reconstructing a target signal X, that is, the fluorescence molecular spatial, spectral and intensity distribution information of the labeled target nucleic acid sequence, thereby realizing efficient nucleic acid testing and gene sequencing.

### BACKGROUND OF THE INVENTION

### 1. Field of the Invention

The present invention relates to the field of molecular diagnosis, more particularly to an efficient nucleic acid testing and gene sequencing method and apparatus.

### 2. Related Art

Nucleic acid and gene detection mainly adopt polymerase chain reaction (PCR) technology, which is a kind of technology that specifically amplifies target DNA sequence in vitro. Through the denaturation, annealing and extension of multiple cycles, trace genetic substances are amplified millions of times within a few hours, and are then characterized or quantified by detecting the fluorescent signal after PCR amplification. PCR technology has become the most important supporting technology and core driving force in the fields of life science research and clinical molecular diagnosis. PCR technology mainly includes real-time quantitative PCR (qPCR) technology and digital PCR (dPCR) technology. However, the current PCR technology is still unable to meet the market demand for fast and efficient nucleic acid test in terms of high accuracy, low concentration and rapid detection. In order to improve the efficiency of nucleic acid test, commercial dPCR adopts sample segmentation technology to prepare tens of thousands or even millions of parallel PCR reaction units, or optimizes the PCR reaction processes and increases the number of fluorescent marked channels (multiplex PCR) to achieve high throughput and highly sensitive detection. However, it still cannot solve the problem of low detection efficiency caused by long PCR reactions.

Gene sequencing is a technique for detecting the nucleic acid sequence of living body according to the principle of base complementary pairing, including DNA sequencing and RNA sequencing, and the method of fluorescent labeling is generally used to carry out gene sequencing, by labeling four kinds of bases with four different fluorescence marks to identify bases through four-color fluorescence detection. In order to improve the efficiency of gene sequencing, the gene sequencer has also undergone three generations of development. The technical basis of the second-generation sequencer is the fluorescence imaging of high-density gene chips, the advantages thereof are high throughput and low cost, and the disadvantage thereof is that the library preparation process of DNA amplification must be realized by polymerase chain reaction (PCR) before sequencing, which may introduce exogenous base mutations, and the second-generation sequencing technology generally is limited by a short reading length of genes. Because the third-generation sequencing technology does not require the process of preparing a library by PCR, it can directly sequence the DNA molecules in the sample, and thus it has potential advantages of fast speed and high accuracy, and it is expected to greatly reduce costs. However, limited to the current level of technological development, the error rate of sequencing is still relatively high, and the throughput and cost cannot be compared with the second-generation sequencing technology within a short period.

Therefore, the current nucleic acid, gene detecting and sequencing technology still have an insurmountable bottleneck due to the limitation of traditional optical detection technology, mainly embodied in the following two aspects:

1) Multi-channel detection efficiency: by labeling with different fluorescent dyes, data from different channels can be obtained, and the detection category from one sample can be improved. However, due to the limitation of optical detection methods, the current multi-channel implementation methods on the market mainly include the following two detection methods: 1. a multi-channel switching sequential exposure detection method, only one fluorescent reagent can be detected at a time, and the detection efficiency is low, which cannot meet the purpose of detecting multiple fluorescent reagents at the same time; 2. The single-channel system is mechanically superimposed or integrated, and each independent detection system and each separate reagent to be detected are connected by optical fibers, the detection efficiency is improved as compared with the single-channel system, but the whole system is larger in size and higher in cost. However, the disadvantage of the above two methods is that they need to perform a step-by-step detection on multiple fluorescence channels, the sample is repeatedly illuminated or the illumination time is different, and the influence of fluorescence quenching is difficult to evaluate. In addition, there is a large crosstalk and interference between different fluorescence channel detection, and thus the multicolor fluorescence detection technique is defective.

2) Detection time: the current optical detection methods cannot avoid long-time PCR process to accumulate fluorescence signal due to the less sensitivity of optical detection, that is, the optical detection capability determines the required cycle number of PCR. The current long detection time is due to insufficient optical detection capability, and thus results in too many PCR cycles required, which essentially cannot meet the requirements of rapid detection. Therefore, improving the sensitivity of fluorescence detection is very important to achieve rapid PCR. By realizing weak light detection, the requirement of PCR amplification cycles can be greatly reduced. To achieve weak light detection, on the one hand, we can start from the sample preparation to achieve high signal-to-noise ratio, high purity, and high brightness fluorescent probes labeling. On the other hand, optimizing the optical system and improving the detection sensitivity of weak fluorescent signals are more essential solutions.

The method that improves detection sensitivity at present mainly contains following two kinds: 1. using kinds of optical structures of confocal type and optical fiber type, their advantages are simple structure, high fluorescence collection efficiency, and higher detection accuracy. However, due to the point scanning method, the imaging time is long, especially for multi-channel detection, the scanning method greatly reduce the detection time. In addition, this method has high requirements of the stability of the light source and the system, and thus it is necessary to avoid the fluctuation of the luminous intensity and the system instability. 2. Large field of view, high-throughput objectives are designed to improve fluorescence collection efficiency, but this often results in expensive equipment.

Therefore, due to the limitation of traditional optical detection technique, the information acquisition efficiency is low, thereby the detection sensitivity and throughput are restricted, which leads the low detection efficiency.

### SUMMARY OF THE INVENTION

The purpose of the present invention is to provide an efficient nucleic acid testing and gene sequencing method and apparatus, thereby solving the problems of low detection sensitivity, low throughput and low detection efficiency of the nucleic acid testing and gene sequencing methods in the prior art.

In order to solve above-mentioned technical problems, the present invention adopts following technical solutions:

According to the first aspect of the present invention, an efficient nucleic acid testing and gene sequencing method is provided, comprising the steps of S1: constructing a space and spectral calibration matrix A as a prior information; S2: labeling a target nucleic acid sequence with fluorescent probes to prepare a nucleic acid chip with a spatial distribution, and exciting the nucleic acid chip with a light source to emit multicolor fluorescent signals, and sequentially modulating, encoding and collecting the multicolor fluorescent signals with an imaging module and an area array detector, and thus obtaining a fluorescence two-dimensional intensity measurement matrix Y; and S3: performing correlation calculation between the calibration matrix A and the measurement matrix Y through a correlation imaging algorithm, solving Y=AX, and reconstructing a target signal X, that is, the fluorescence molecular spatial, spectral and intensity distribution information of the labeled target nucleic acid sequence, thereby realizing efficient nucleic acid testing and gene sequencing.

The operating principle of above-mentioned method according to the invention is: labeling target nucleic acid sequences with different fluorescent probes, exciting the nucleic acid chip with the light source, processing and collecting the multi-color fluorescent signals, and reconstructing the collected signals with optical correlation imaging method to obtain the spatial, spectral and intensity distribution information of the labeled fluorescent probes, and thus to achieve high-throughput, high-sensitivity, fast, and multiplex nucleic acid testing and gene sequencing.

In the step S1, the spatial and spectral calibration matrix A is obtained through experimental calibration or ray tracing and wave optics calculation, or through deep learning training. The spatial and spectral calibration matrix A is constructed by the light intensity distribution of point light sources with different spatial positions and different wavelengths on the calibrating surface being imaged by the imaging module on the area array detector.

According to a preferred embodiment of the present invention, in the step S2, the imaging module comprises projection lens sets and multi-channel filter sets, and what the area array detector detects is a two-dimensional intensity measurement matrix of polychromatic fluorescence based on point spread function, Gaussian spot or Airy disk.

According to another preferred embodiment of the present invention, in the step S2, the imaging module comprises projection lens sets, multi-channel filter sets and a spatial modulation module, and wherein the spatial modulation module adopts spatial random phase modulator to realize random modulation of the light field to obtain a speckle image of the fluorescence signal, and what the area array detector detects is a two-dimensional intensity measurement matrix of polychromatic fluorescence based on the speckle image.

According to another preferred embodiment of the present invention, in the step S2, the imaging module comprises projection lens sets, multi-channel filter sets and a spatial coding module, wherein the spatial coding module adopts liquid crystal spatial light modulator or DMD to construct a specific two-dimensional encoding matrix, and what the area array detector detects is the encoded two-dimensional intensity measurement matrix of polychromatic fluorescence.

According to another preferred embodiment of the present invention, in the step S2, the imaging module comprises projection lens sets, multi-channel filter sets and a dispersive element, wherein the dispersive element splits the spectral dispersion of the polychromatic fluorescent signal, and what the area array detector detects is a two-dimensional intensity measurement matrix of polychromatic fluorescence based on spectral signals.

In the step S3, as to the detected two-dimensional intensity information of polychromatic fluorescence, including speckle signal, Gaussian spot or Airy disk signal, coding information and spectral information, the correlation imaging algorithm is selected from any one of the following methods:
1) The compressed sensing algorithm: combining with the matrix mapping theory and the optical correlation imaging algorithm, and finding the optimal solution of the signal through, it can quickly recover the spatial and spectral intensity information of the target signal;
2) Deep learning algorithm: by building a neural network model, and using the weak fluorescence signal at different photon number levels to continuously train and optimize the network, so as to achieve the recovery of fluorescence weak signal images;
3) The maximum likelihood estimation algorithm: through the statistical probability relationship between the weak signal and the strong signal, establishing the likelihood function between the weak signal and the signal that needs to be restored, and using the likelihood function to combine with the external prior information of the weak signal to construct the objective function, optimizing the likelihood function through the optimization method, so as to complete the recovery of the weak fluorescence signal, or combine with the compressed sensing algorithm to realize the sparse Poisson-based compressed sensing algorithm;
4) The image reconstruction algorithm based on sparse constraints: combining with the sparse characteristics of labeled fluorescent signals, and the characteristics that noise cannot be sparsely expressed, a sparse constraint is imposed on the signal to be restored, and an optimization problem is constructed combined with the noise variance distribution, and then restoring the original weak fluorescent signal by an optimization algorithm, or combining with the compressed sensing algorithm, so as to realize the compressed sensing algorithm based on sparse constraints.

According to the second aspect of the present invention, an efficient nucleic acid testing and gene sequencing apparatus is provided, for realizing above-mentioned efficient nucleic acid testing and gene sequencing method. The apparatus includes: an excitation light source module, which is a single-channel excitation light source or multiple-channel excitation light sources according to the requirements of single or multiple target fluorescent labeling of nucleic acid samples; an imaging module, including: projection lens sets and multi-channel filter sets; and an area array detector; wherein, after being excited by single-channel excitation light source or multi-channel excitation light source, the nucleic acid sample to be detected emits one-color or multi-color fluorescence signal, which is modulated and encoded by the imaging module, and then sampled by the area array detector, and finally the correlation imaging algorithm is adopted to recover the spatial, spectral and intensity distribution information of one-color or multi-color fluorescent molecules in the sample, so as to realize high-throughput, high-sensitivity, rapid, multiple nucleic acid testing and sequencing of nucleic acid samples.

The nucleic acid sample to be detected can be a real-time quantitative PCR (qPCR) sample or a digital PCR (dPCR) chip for nucleic acid testing, or a gene chip for gene sequencing, etc. Nucleic acid samples can be labeled with various fluorophores according to the detection requirements.

The excitation light source module adopts multi-way or single-way excitation light source according to fluorescence detection requirement. The excitation light source is a high-power, narrowband LED light source or a laser. The excitation light source module has no mechanical apparatus, and has the advantages of simple structure, small volume and high wavelength utilization rate.

The imaging module includes following four kinds:

1) It includes projection lens sets and multi-channel filter sets, wherein the projection lens sets can be realized by large-aperture and short-focus compound lens, or a high numerical aperture objective lens, or projection objective lens, or microlens array, to achieve a large field of view, high-efficiency fluorescence signal collection. The multi-channel filter sets include a dichroic mirror and a filter in front of the detection module. The dichroic mirror is used to reflect the light source into the lens sets to excite the sample, and pass the collected fluorescence from the lens sets. The filter is a multi-channel filter, which suppresses the excitation light source and obtains multi-color fluorescent signals with good signal-to-noise ratio. Through the imaging module, what the area array detector detects is the fluorescence signal Gaussian spot or Airy disk signal.

2) It includes projection lens sets, multi-channel filter sets and a spatial modulation module. The projection lens sets and the multi-channel filter sets are the same as the above 1). The spatial modulation module uses a spatial random phase modulator to realize light field random modulation of the fluorescent signal, and obtains the speckle image of the fluorescent signal. The area array detector detects the speckle image on the entire imaging surface. The spatial random phase modulator is a ground glass with a certain aspect ratio range and random distribution of particles, or a phase modulator that generates random phases under computer programming control.

3) It includes projection lens sets, multi-channel filter sets and a spatial encoding module. The spatial encoding module uses a liquid crystal spatial light modulator or a digital micromirror device (DMD) to construct a specific two-dimensional encoding matrix to encode the intensity of the fluorescent signal, and what the area array detector detects is the intensity information encoded by the fluorescent signal.

4) It includes projection lens sets, multi-channel filter sets and a dispersion element. The fluorescent collection lens sets and the multi-channel filter sets are the same as the above-mentioned first imaging module. The dispersive element adopts a grating or a prism to realize spectral dispersion and light separation, and what the area array detector detects is the fluorescent signal spatial and spectral information.

The area array detector adopts the single photon camera that is formed by the combination of image intensifier and high-speed CMOS camera, or a two-dimensional array of photomultiplier tube (PMT)/avalanche diode (APD), which has nanosecond high-speed electron shutter and picosecond-level high-precision timing control, and can achieve high-speed detection with single-photon sensitivity, while effectively suppressing the interference of background light. Other highly sensitive CMOS or CCD detectors can also be used.

The present invention provides an efficient nucleic acid testing and gene sequencing method, which adopts fluorescent probes to label target nucleic acid sequence, excites the nucleic acid chip with a light source and collects fluorescent signal, utilizes correlation imaging method to reconstruct the spatial, spectral and intensity distribution information collected fluorescent signal of labeled fluorescent probes, so as to realize high-throughput, high-sensitivity, rapid, multiplex nucleic acid gene detection and sequencing. Based on the method, an efficient nucleic acid testing and gene sequencing apparatus is provided. The invention applies the image reconstruction method and algorithm based on correlation imaging to nucleic acid and gene detection and sequencing, and utilizes a prior information to greatly improve the efficiency of information acquisition, as well as the signal-to-noise ratio, reconstruction accuracy and speed of image restoration, thereby shortens nucleic acid, gene detection and sequencing time, and improves nucleic acid testing efficiency, detection sensitivity, throughput and accuracy.

As compared with existing method and apparatus, the efficient nucleic acid testing and gene sequencing method and apparatus of the present invention have the following advantages:

1) High-sensitivity and rapid detection. Using the correlation imaging method and prior information, can greatly improve signal-to-noise ratio of the image restoration and reconstruction accuracy. Combining with the single-photon sensitivity camera, the detection sensitivity and the acquisition efficiency of image information can be improved; at the same time, it can efficiently detect fluorescent signals in fewer PCR cycles, and shorten detection time.

2) Multiple fluorescence rapid detection. Using random phase modulator, coding and dispersive elements, combining with the correlation imaging reconstruction method, it can realize single-exposure multi-color fluorescence imaging. It overcomes the limitation of slow detection speed due to the multi-channel switching sequential exposure method used in traditional multiplex fluorescent PCR detection and gene sequencing, and eliminates the problem of interference between fluorescent signals. Thus, real-time synchronous and rapid detection of multi-sample and multi-gene targets is realized, the detection efficiency is improved, and the detection cost is reduced.

3) Quantitative detection of low-concentration nucleic acid. The present invention adopts a highly sensitive and high-throughput detection method, which breaks through the limitations of traditional nucleic acid testing and gene sequencing on low-concentration nucleic acid samples, and has revolutionary advantages in low-concentration nucleic acid testing.

4) Miniaturization of the instrument. The optical system of the present invention has a simple structure and no mechanical transmission apparatus, which simplifies the complex opto-mechanical apparatus required for multi-channel fluorescence switching of traditional PCR instruments and gene sequencing apparatuses, and simplifies the optical system designed for achieving high throughput. The system is conducive to realize miniaturization.

Above all, the present invention changes the traditional optical detection mode, develops an optical correlation imaging method for nucleic acid testing and gene sequencing, and proposes a small and efficient nucleic acid testing and gene sequencing apparatus based on the method, thereby achieves high-throughput, high-sensitivity, rapid, multiplex nucleic acid testing and gene sequencing.

### BRIEF DESCRIPTION OF DRAWINGS

FIG. 1 is a principle and flow diagram of an efficient nucleic acid testing and gene sequencing method according to the present invention.

FIG. 2 is a principle schematic diagram of a small and efficient nucleic acid testing and gene sequencing apparatus based on a spatial phase modulator according to an embodiment of the present invention.

FIG. 3 is a principle schematic diagram of a small and efficient nucleic acid testing and gene sequencing apparatus based on a dispersive element according to another embodiment of the present invention.

FIG. 4 is a principle schematic diagram of a small and efficient nucleic acid testing and gene sequencing apparatus based on a spatial coding module according to yet another embodiment of the present invention.

FIG. 5 is a principle schematic diagram of a small and efficient nucleic acid testing and gene sequencing apparatus according to yet another embodiment of the present invention.

### DESCRIPTION OF THE ENABLING EMBODIMENT

Embodiments of the present invention are described below by specific embodiments, thus those skilled in the art can easily understand other advantages and effects of the present invention from the content disclosed in this specification. The present invention can also be implemented or applied through other different specific embodiments, and various details in this specification can also be modified or changed based on different viewpoints and applications without departing from the spirit of the present invention. It should be noted that the following embodiments and features in the embodiments may be combined with each other without conflict.

It should be noted that the illustrations provided in the following embodiments only illustrate the basic conception of the present invention in a schematic way, thus the drawings only show the components related to the present invention, instead of drawing according to the number, shape and size of the components in actual implementation. The type, number and ratio of each component in actual implementation can be changed at will, and its component layout patterns may also be more complex.

### First Embodiment: Efficient Nucleic Acid Testing and Gene Sequencing Method

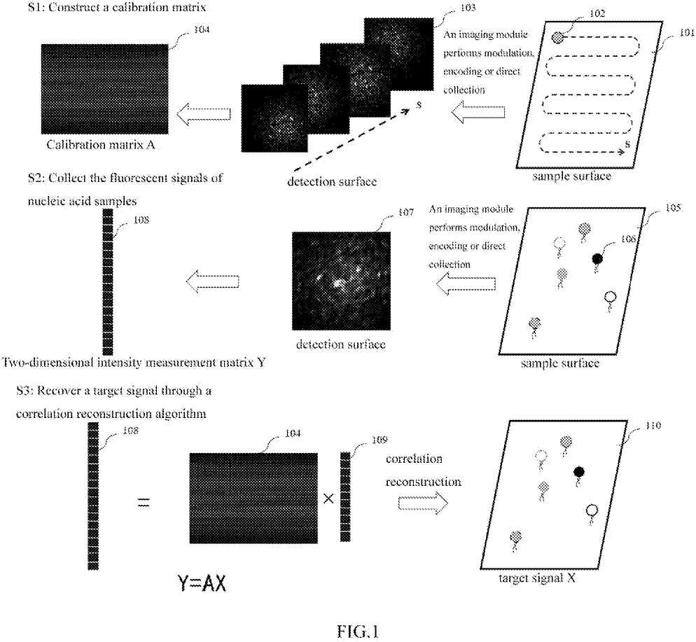

As shown in FIG. 1, an efficient nucleic acid testing and gene sequencing method according to the first embodiment of the present invention comprises the following steps:

Step S1: the fluorescence signal of a single wavelength λ 1 emitted by the point light source 102 on the sample surface 101 is modulated or encoded by the imaging module or directly imaged on the detector, and the two-dimensional fluorescence intensity information 103 detected by the detector is used as a column A(i,1) of a calibration matrix A104; then the point light source 102 is moved to the next position on the sample surface 101, the intensity information of this position is obtained as A(i,2), and this operation is repeated until the intensity information 103 of the point light source at each position on the sample surface 101 is obtained, which is used as the calibration matrix A(i,1)~A(i,n); another point light source with a wavelength λ 2 is selected, and the above operation is repeated to obtain the intensity information of the point light source on the detection surface under the different wavelengths λ 1 ∼λ m and at different spatial positions, a spatial and spectral calibration matrix A i×j is constructed, wherein i is the number of pixels of the two-dimensional detector, and j is m×n. This process can be obtained through experimental calibration or ray tracing and wave optics calculations, or deep learning training.

Step S2: The different fluorescent probes are adopted to label target nucleic acid sequence 106 to prepare a nucleic acid chip 105 with spatial distribution, the nucleic acid chip 105 is excited to send out multicolor fluorescence signal, imaging module and area array detector are adopted to modulate, encode and collect it successively, so that multi-color fluorescence intensity information 107 is obtained, and a two-dimensional intensity measurement matrix Y108 is constructed.

Step S3: The calibration matrix 104 and the measurement matrix 108 are correlated and calculated by the correlation imaging algorithm to solve Y=AX, and the target signal X109 is reconstructed, that is, the fluorescent molecule space, spectral and intensity distribution information of the labeled target nucleic acid sequence in the nucleic acid chip 105.

It should be noted that, in the steps S1 and S2, the imaging module includes following four kinds:

1. It includes projection lens sets and multi-channel filter sets, and what the area array detector detects is polychromatic fluorescence two-dimensional intensity measurement matrix based on point spread function, Gaussian spot or Airy disk.

2. It includes projection lens sets, multi-channel filter sets and a spatial modulation module. The spatial modulation module uses a spatial random phase modulator to realize light field random modulation, so as to obtain the speckle image of the fluorescent signal. What the area array detector detects is a polychromatic fluorescence two-dimensional intensity measurement matrix based on the speckle image.

3. It includes projection lens sets, multi-channel filter sets and a spatial encoding module. The spatial encoding module uses a liquid crystal spatial light modulator or a digital micromirror device (DMD) to construct a specific two-dimensional encoding matrix, and what the area array detector detects is the encoded polychromatic fluorescence two-dimensional intensity measurement matrix.

4. It includes projection lens sets, multi-channel filter sets and a dispersion element. The dispersive element disperses the spectrum of the polychromatic fluorescence signal, and what the area array detector detects is a polychromatic fluorescence two-dimensional intensity measurement matrix based on spectral signals.

Therefore, in the steps S1 and S2, the detected two-dimensional fluorescence intensity information 103 and the multicolor fluorescence intensity information 107 can be the multicolor fluorescence two-dimensional intensity based on point spread function, Gaussian spot or Airy disk information, or multicolor fluorescence two-dimensional intensity information based on speckle image, or encoded multicolor fluorescence two-dimensional intensity information, or multicolor fluorescence two-dimensional intensity information based on spectral signals.

It should be noted that, in the step S3, the correlation imaging algorithm specifically includes the following four kinds:
1) The compressed sensing algorithm: by making full use of the developed compressed sensing theory and algorithm, combining with the matrix mapping theory and algorithm, the measured fluorescence molecular space and spectral intensity information can be quickly recovered;
2) Deep learning algorithm: by constructing neural network models, including convolutional neural networks, fully connected networks, generative adversarial networks and their combinations, and by using weak fluorescence signals at different photon number levels to continuously train and optimize the network, the restoration of fluorescence weak signal images is achieved;
3) The maximum likelihood estimation algorithm: through the statistical probability relationship between the weak signal and the strong signal, establishing the likelihood function between the weak signal and the signal that needs to be restored, and using the likelihood function to combine with the external prior information of the weak signal to construct the objective function, the maximum likelihood function is achieved through the optimization method, and then combining with the compressed sensing algorithm to complete the restoration of the original signal;
4) The image reconstruction algorithm based on sparse constraints: combining with the sparse characteristics of labeled fluorescent signals, and the characteristics that noise cannot be sparsely expressed, a sparse constraint is imposed on the signal to be restored, and an optimization problem is constructed combined with the noise variance distribution, and then restoring the original weak fluorescent signal by an optimization algorithm, or combining with the compressed sensing algorithm, so as to realize the compressed sensing algorithm based on sparse constraints.

### Second Embodiment: Small and Efficient Nucleic Acid Testing and Gene Sequencing Apparatus Based on Spatial Phase Modulator

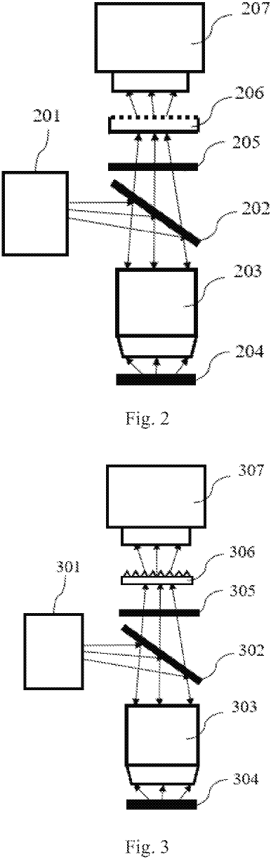

FIG. 2 shows a small and efficient nucleic acid testing and gene sequencing apparatus according to the second embodiment of the present invention, including an excitation light source module 201, an imaging module 202-206, and an area array detector 207. The multi-channel or single-channel excitation light source 201 is reflected by a dichroic mirror 202, and illuminates a nucleic acid sample 204 to be detected through projection lens sets 203, and the fluorescent signal generated by the excitation of the sample passes through the projection lens sets 203 and then the dichroic mirror 202, a multi-channel filter sets 205 further filters out the interference of the excitation light source, and a spatial phase modulator 206 performs random modulation of the light field on the fluorescence signal to obtain a speckle image of the fluorescence signal. Then the area array detector 207 is used to sample the speckle image on the entire imaging surface, and finally the correlation imaging algorithm is used to recover the spatial, spectral and intensity distribution information of the fluorescent molecules in the sample, so as to realize high-throughput and rapid multiple nucleic acid testing and gene sequencing.

In this embodiment, the excitation light source module 201 adopts a multi-channel or single-channel LED light source or laser.

In this embodiment, the nucleic acid sample 204 to be detected can be a real-time quantitative PCR (qPCR) sample, or a digital PCR (dPCR) chip for nucleic acid testing, or a gene chip for gene sequencing, etc. Nucleic acid samples can be labeled with various fluorophores according to the detection requirements.

In this embodiment, the projection lens sets 203 can be a compound lens with a large aperture and short focus, or a high numerical aperture objective lens or a projection objective lens, or a microlens array, so as to realize a large field of view and high-efficiency fluorescence signal collection.

The spatial random phase modulator 206 is ground glass with a certain aspect ratio range and random distribution of particles, or a phase modulator that generates random phases under computer programming control.

In this embodiment, the area array detector 207 can be a single-photon camera composed of a combination of an image intensifier and a high-speed CMOS camera, or a two-dimensional array of photomultiplier tubes (PMT)/avalanche diodes (APD), with nanosecond high-speed electronic shutter and picosecond high-precision timing control.

In this embodiment, as the area array detector detects the fluorescence speckle signal, the fluorescence weak signal image reconstruction algorithm is correlation imaging algorithm mainly based on speckle field, and the specific algorithm refers to step S3 in the first embodiment.

Thus, in the present embodiment, the advantage of the optical correlation imaging algorithm based on random measurement of speckle field is that the random measurement method improves the random characteristic of the signal by performing light field random modulation on the fluorescent signal, it can better meet the requirements of compressive sensing random measurement, greatly improve the positioning accuracy and density of signal reconstruction, and has spectral resolution capability to achieve single-exposure multicolor imaging. This method greatly improves the efficiency of information acquisition, and can achieve fast, high-throughput, highly sensitive nucleic acid testing and gene sequencing.

### Third Embodiment: Small and Efficient Nucleic Acid Testing and Gene Sequencing Apparatus based on dispersive element

FIG. 3 shows a small and efficient nucleic acid testing and gene sequencing apparatus according to a third embodiment of the present invention, including an excitation light source module 301, an imaging module 302-306, and an area array detector 307. The multi-channel or single-channel excitation light source 301 is reflected by a dichroic mirror 302, and illuminates a nucleic acid sample 304 to be detected through projection lens sets 303, and the fluorescent signal generated by the excitated sample passes through the projection lens sets 303 and then through the dichroic mirror 302, a multi-channel filter sets 305 further filters out the interference of the excitation light source, and a dispersive element 306 performs spectral dispersion on the fluorescence signal to obtain the spectral information of the fluorescence signal. The area array detector 307 is then used to sample the spatial and spectral information of the fluorescence signal on the entire imaging surface, and finally the correlation imaging algorithm is used to recover the spatial, spectral and intensity distribution information of the fluorescent molecules in the sample, so as to achieve high-throughput, rapid multiplex nucleic acid testing and gene sequencing.

In this embodiment, the excitation light source module 301, multi-channel filter sets 302, the projection lens sets 303, the nucleic acid sample 304 to be tested, and the area array detector 307 are the same as the first embodiment.

In this embodiment, the dispersive element 306 may be a grating or a prism.

In this embodiment, as the area array detector detects the fluorescence signal space and spectral information, the correlation imaging algorithm is mainly based on the correlation imaging calculation of space and spectral signals, and the specific algorithm refers to step S3 in the first embodiment.

### Fourth Embodiment: a Small and Efficient Nucleic Acid Testing and Gene Sequencing Apparatus Based on the Spatial Coding Module

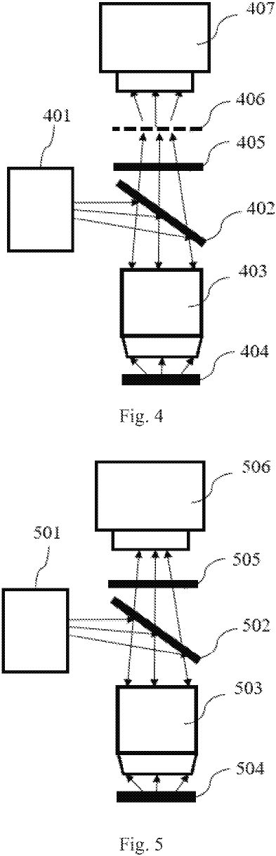

FIG. 4 shows a small and highly sensitive multiple nucleic acid rapid detector according to the fourth embodiment of the present invention, including an excitation light source module 401, an imaging module 402-406, and an area array detector 407. The multi-channel or single-channel excitation light source 401 is reflected by a dichroic mirror 402, and illuminates a nucleic acid sample 404 to be detected through projection lens sets 403, and the fluorescent signal generated by the excited sample passes through the projection lens sets 403 and then the dichroic mirror 402, multi-channel filter sets 405 further filters out the interference of the excitation light source, and a spatial encoding module 406 performs spatial intensity encoding on the fluorescence signal. Then the area array detector 407 is used to sample the encoded fluorescent signal spatial information on the entire imaging surface, and finally the correlation imaging algorithm is used to recover the spatial, spectral and intensity distribution information of the fluorescent molecules in the sample, so as to achieve high-throughput, rapid multiplex nucleic acid testing and gene sequencing.

In this embodiment, the excitation light source module 401, the multi-channel filter sets 402, the projection lens sets 403, the nucleic acid sample 404 to be detected, and the area array detector 407 are the same as the first embodiment.

In this embodiment, the spatial encoding module 406 adopts a liquid crystal spatial light modulator or DMD to construct a specific two-dimensional encoding matrix.

In the present embodiment, as the area array detector detects the spatial information encoded by the fluorescent signal, the correlation imaging algorithm is mainly based on the correlation imaging calculation of the encoded spatial signal, and the specific algorithm refers to the steps S3 in the first embodiment.

### Fifth Embodiment: Small and Efficient Nucleic Acid Testing and Gene Sequencing Apparatus

FIG. 5 shows a small and highly sensitive multiple nucleic acid rapid detector according to the fifth embodiment of the present invention, including an excitation light source module 501, an imaging module 502-505, and an area array detector 506. The multi-channel or single-channel excitation light source 501 is reflected by a dichroic mirror 502, and illuminates a nucleic acid sample 504 to be detected through projection lens sets 503, and the fluorescent signal generated by the excited sample passes through the projection lens sets 503 and then the dichroic mirror 502, and the multi-channel filter sets 505 further filters out the interference of the excitation light source. A fluorescence signal image based on point spread function (PSF), Gaussian spot or Airy disk is obtained, and the area array detector 506 is used to directly sample the fluorescence signal on the entire imaging surface, and finally the correlation imaging algorithm is used to restore the spatial, spectral and intensity distribution information of fluorescent molecules in the sample, so as to realize high-throughput and rapid multiplex nucleic acid testing and gene sequencing.

In this embodiment, the excitation light source module 501, the projection lens sets 503, the multi-channel filter sets 502, the nucleic acid sample 504 to be detected, and the area array detector 506 are the same as the first embodiment.

In this embodiment, as the area array detector detects the point spread function (PSF), Gaussian spot or Airy (Airy) spot of the fluorescent signal spatial distribution, the correlation imaging algorithm is mainly based on the correlation imaging calculation of the point spread function, Gaussian spot or Airy spot, and the specific algorithm refers to the step S3 in the first embodiment.

The above are only preferred embodiments of the present invention, and are not intended to limit the scope of the present invention, and various changes can also be made in the above-described embodiments of the present invention. All simple and equivalent changes and modifications made according to the claims and descriptions of the present application shall fall within the protection scope of the claims of the present invention. What is not described in detail in the present invention is conventional technical content.

1. An efficient nucleic acid detection and gene sequencing method, characterized in that, comprising the following steps:
S0: providing an efficient nucleic acid detection and gene sequencing device, including: an excitation light source module, which is a single-channel excitation light source or multiple-channel excitation light sources according to the requirements of single or multiple target fluorescent labeling of nucleic acid samples; an imaging module, including: projection lens sets and multi-channel filter sets; and a spatial modulation module, or a spatial coding module, or a dispersive element, wherein the spatial modulation module adopts spatial random phase modulator to realize random modulation of the light field to obtain a speckle image of the fluorescence signal, and the spatial coding module adopts liquid crystal spatial light modulator or DMD to construct a specific two-dimensional encoding matrix; and an area array detector, which adopts a single photon camera composed of a microchannel plate-based image intensifier and a high-speed CMOS camera, or a two-dimensional array of photomultiplier tubes/avalanche diodes, or a highly sensitive area array CMOS or CCD camera;
S1: constructing a space and spectral calibration matrix A as a prior information;
S2: labeling a target nucleic acid sequence with fluorescent probes to prepare a nucleic acid chip with a spatial distribution, and exciting the nucleic acid chip with the light source to emit multicolor fluorescent signals, and sequentially modulating, encoding and collecting the multicolor fluorescent signals with the imaging module and the area array detector, and thus obtaining a fluorescence two-dimensional intensity measurement matrix Y; and
S3: performing correlation calculation between the calibration matrix A and the measurement matrix Y through a correlation imaging algorithm, solving Y=AX, and reconstructing a target signal X, that is, the fluorescence molecular spatial, spectral and intensity distribution information of the labeled target nucleic acid sequence, thereby realizing efficient nucleic acid detection and gene sequencing.

2. The efficient nucleic acid detection and gene sequencing method according to the claim 1, wherein, in the step S1, the spatial and spectral calibration matrix A is obtained through experimental calibration or ray tracing and wave optics calculation, or through deep learning training, the spatial and spectral calibration matrix A is constructed by the light intensity distribution of point light sources with different spatial positions and different wavelengths on the calibrating surface being imaged by the imaging module on the area array detector.

3. The efficient nucleic acid detection and gene sequencing method according to the claim 1, wherein, in the step S2, the imaging module comprises projection lens sets and multi-channel filter sets, and what the area array detector detects is polychromatic fluorescence two-dimensional intensity measurement matrix based on point spread function, Gaussian spot or Airy disk.

4. The efficient nucleic acid detection and gene sequencing method according to the claim 1, whererin, in the step S0 and S2, the imaging module comprises projection lens sets, multi-channel filter sets and a spatial modulation module, and wherein the spatial modulation module adopts spatial random phase modulator to realize random modulation of the light field to obtain a speckle image of the fluorescence signal, and what the area array detector detects is a polychromatic fluorescence two-dimensional intensity measurement matrix based on the speckle image.

5. The efficient nucleic acid detection and gene sequencing method according to the claim 1, wherein, in the step S0 and S2, the imaging module comprises projection lens sets, multi-channel filter sets and a spatial coding module, wherein the spatial coding module adopts liquid crystal spatial light modulator or DMD to construct a specific two-dimensional encoding matrix, and what the area array detector detects is the encoded polychromatic fluorescence two-dimensional intensity measurement matrix.

6. The efficient nucleic acid detection and gene sequencing method according to the claim 1, wherein, in the step S0 and S2, the imaging module comprises projection lens sets, multi-channel filter sets and a dispersive element, wherein the dispersive element splits the spectral dispersion of the polychromatic fluorescent signal, and what the area array detector detects is a polychromatic fluorescence two-dimensional intensity measurement matrix based on spectral signals.

7. The efficient nucleic acid detection and gene sequencing method according to the claim 1, wherein, in the step S3, the correlation imaging algorithm is selected from any one of following four kinds:
1) The compressed sensing algorithm: combining with the matrix mapping theory and the optical correlation imaging algorithm, finding the optimal solution of the signal through
$${min}_{x} {\left‖X\right‖}_{{l}_{1}} s u b j e c t t o Y = A X ,$$
it can quickly recover the spatial and spectral intensity information of the target signal;
2) Deep learning algorithm: by constructing neural network models, and by using weak fluorescence signals at different photon number levels to continuously train and optimize the network, so as to achieve the restoration of fluorescence weak signal images;
3) The maximum likelihood estimation algorithm: through the statistical probability relationship between the weak signal and the strong signal, establishing the likelihood function between the weak signal and the signal that needs to be restored, and using the likelihood function to combine with the external prior information of the weak signal to construct the objective function, optimizing the likelihood function by the optimization method, so as to complete the recovery of the weak fluorescence signal, or combining with the compressed sensing algorithm to realize the sparse Poisson-based compressed sensing algorithm; and
4) The image reconstruction algorithm based on sparse constraints: combining with the sparse characteristics of labeled fluorescent signals, and the characteristics that noise cannot be sparsely expressed, a sparse constraint is imposed on the signal to be restored, and an optimization problem is constructed combined with the noise variance distribution, and then restoring the original weak fluorescent signal by an optimization algorithm, or combining with the compressed sensing algorithm, so as to realize the compressed sensing algorithm based on sparse constraints.

8. The efficient nucleic acid detection and gene sequencing method according to the claim 1, wherein, in the step S0, the excitation light source is LED or laser.

9. The efficient nucleic acid detection and gene sequencing method according to the claim 1, wherein, in the step S0, the projection lens sets includes: large-aperture and short-focus compound lens, or high numerical aperture objective lens, or projection objective lens, or microlens array.
